(19) United States
(12) Patent Application Publication
(10) Pub. No.: US 2016/0143729 A1
(43) Pub. Date: May 26, 2016

(54) Regenerative Respiratory Tract Prostheses

(71) Applicant: CorMatrix Cardiovascular, Inc., Roswell, GA (US)

(72) Inventor: Robert G. Matheny, Norcross, GA (US)

(21) Appl. No.: 14/554,672

(22) Filed: Nov. 26, 2014

(51) Int. Cl. A61F 2/20; A61L 27/54; A61L 27/16; A61L 27/20; A61L 27/36; A61L 27/58

(52) U.S. Cl. CPC A61F 2/203; A61L 27/3633; A61L 27/58; A61L 27/16; A61L 27/20; A61L 27/54; A61F 2210/0004

(57) ABSTRACT

A regenerative respiratory tract prosthesis comprising a tubular extracellular matrix (ECM) member having a lumen therethrough, at least first and second biodegradable polymeric anchors disposed on the tubular member exterior surface. The first and second anchors can include a microneedle tissue engagement member.

### FIELD OF THE INVENTION

The present invention relates to methods and apparatus for repairing damaged, diseased or missing respiratory tract segments. More particularly, the present invention relates to regenerative respiratory tract prostheses for treating and/or repairing damaged, diseased and/or missing tracheal and/or bronchial segments.

### BACKGROUND OF THE INVENTION

As is well known in the art, the trachea is an azygous organ—the proper function of which is of critical importance for the human organism. As illustrated in FIG. 1, the trachea 10 is located between the larynx 14 in the upper respiratory tract 12 and the primary bronchi 18 of the lungs 20 in the lower respiratory tract 16. The function of the trachea 10 is to deliver oxygen to the lungs 20 and filter foreign substances via the cilia on the pseudostratified ciliated columnar epithelium (PCCE) 34, which, as illustrated in FIG. 2, lines the lumen 32 of the trachea 10.

As is also well known in the art, damage or disease of the PCCE; particularly, where the damage or disease results in a missing segment or portion of the PCCE, can, and often will, induce severe adverse physiological consequences, e.g., reduced immunity.

Various conventional devices and methods have thus been developed to treat and/or repair damaged, diseased and/or missing PCCE segments.

If the trachea and, hence, is damaged only over a short or a moderate length, e.g., 2 cm to 4 cm, a method often employed is to resect the affected segment. The remaining tracheal stumps are then connected by suturing.

If the trachea is damaged only over a longer length, skin and cartilage grafting is typically employed.

There are several significant drawbacks associated with the noted surgical methods. A major drawback is the formation of scar tissue and absorption of the grafted cartilage in the newly formed respiratory tract, which can, and often will, constrict the tract.

More recently, artificial trachea apparatus or prostheses have been employed to repair damaged, diseased and/or missing trachea segments. Illustrative are the trachea prostheses disclosed in U.S. Pat. Nos. 5,486,431 and 5,258,027.

The trachea prostheses disclosed in U.S. Pat. Nos. 5,486,431 and 5,258,027 comprise polymer shafts with metal clasps (i.e. '431 patent) and polymer hoops (i.e. '027 patent) that are disposed circumferentially around the shafts.

There are, however, several significant drawbacks and disadvantages associated with conventional polymeric prostheses. A common problem is that the outer surface of the prostheses are typically very smooth, which can, and often will, result in the prosthesis being dislodged. Such dislodgement has been associated with severe damage to neighboring organs and fistulae between the oesophagus and trachea.

Further, the polymeric materials employed to construct the prostheses, such as poly(ethylene terephthalate) (PET), can, and often will, cause irritation and undesirable biologic responses from the surrounding tissues in the trachea.

In such instances, the prosthesis often must be removed through a secondary surgical procedure, which can, and in many instances will, result in undesirable pain and discomfort to the patient and possibly additional trauma to the trachea. In addition to the pain and discomfort, the patient must be subjected to an additional time consuming and complicated surgical procedure with the attendant risks of surgery.

More recently, biodegradable trachea prostheses have been developed in an effort to eliminate the harsh biological responses associated with conventional polymeric prostheses. There are, however, several known disadvantages associated with biodegradable prostheses.

One major disadvantage is that the biodegradable materials and, hence, prostheses formed therefrom often break down at a faster rate than is desirable for the application. A further disadvantage is that the biodegradable materials can, and in many instances will, break down into large, rigid fragments that can cause obstructions in the respiratory tract.

A further disadvantage associated with conventional trachea prostheses is that existing means for securing the prosthesis in the trachea have had limited success. Often the securing means comprises engaging the prosthesis to the trachea by physical or mechanical means. Another securing means comprises modifying the prosthesis surface or material to induce the production of fibrous (scar) tissue to anchor the prosthesis upon implantation within the vessel.

There is thus a need to provide respiratory tract prostheses that substantially reduce or eliminate the drawbacks and disadvantages associated with conventional apparatus and methods to repair damaged, diseased and/or missing respiratory tract regions, e.g., trachea and/or PCCE segments.

There is also a need to provide respiratory tract prostheses that substantially reduce or eliminate the harsh biological responses associated with conventional polymeric and metal prostheses.

There is also a need to provide respiratory tract prostheses that substantially reduce or eliminate the formation of inflammation and infection.

There is also a need to provide respiratory tract prostheses that can replace or improve biological functions or promote the growth of new respiratory tract tissue in a subject.

There is also a need to provide respiratory tract prostheses that include effective trachea securing means.

There is also the need to provide respiratory tract prostheses having mechanical compatibility with respiratory tract structures and/or enhanced mechanical properties. As is well known in the art, a mismatch between the stiffness, hardness, and porosity of a prosthesis in comparison to the surrounding tissue environment can cause irritation and other complications after implantation.

It is therefore an object of the present invention to provide regenerative respiratory tract prostheses that substantially reduce or eliminate the harsh biological responses associated with conventional polymeric (and metal) prostheses, and the formation of biofilm, inflammation and infection.

It is another object of the present invention to provide regenerative respiratory tract prostheses that can effectively replace or improve biological functions or promote the growth of new tissue in respiratory tract structures.

It is another object of the present invention to provide regenerative respiratory tract prostheses that include effective anchoring means for positioning the prostheses proximate target tissue in a respiratory tract structure.

It is another object of the present invention to provide regenerative respiratory tract prostheses that can administer one or more pharmacological or therapeutic agents to a subject.

### SUMMARY OF THE INVENTION

The present invention is directed to regenerative respiratory tract prostheses and methods using same to treat and/or repair damaged, diseased and/or missing respiratory tract structures; particularly, tracheal and/or bronchial segments.

In a preferred embodiment of the invention, the regenerative respiratory tract prostheses of the invention comprise a tubular shaped member comprising an ECM material (hereinafter referred to as “ECM members”). In a preferred embodiment, the regenerative respiratory tract prostheses further comprise at least one anchoring mechanism.

According to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, omentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof.

The ECM material can also comprise collagen from mammalian sources.

In a preferred embodiment of the invention, the ECM members are capable of transitioning from a pre-deployment configuration, wherein the ECM member is capable of being positioned in a respiratory tract structure, to a post-deployment configuration, wherein the ECM member is disposed proximate host tissue of the respiratory tract structure.

In some embodiments, the anchoring mechanism comprises proximal and distal anchors. In the noted embodiments, the proximal anchor is disposed proximate the proximal end of the ECM member and the distal anchor is disposed proximate the distal end of the ECM member.

In some embodiments, the anchoring mechanism comprises proximal and distal anchors, and at least one mid-region anchor. In the noted embodiments, the proximal anchor is similarly disposed proximate the proximal end of the ECM member and the distal anchor is disposed proximate the distal end of the ECM member. The mid-region anchor (or anchors) is disposed between the proximal and distal anchors.

In a preferred embodiment, the anchors of the invention are configured to temporarily support and position the ECM member and, hence, prosthesis formed therefrom proximate host tissue of a respiratory tract structure, e.g., trachea, and maintain contact therewith throughout the four phases of the ventilatory cycle, i.e. trigger phase, flow delivery phase, cycle phase and expiratory phase, for a predetermined anchor support period of time.

In a preferred embodiment, the anchor support period of time is within the process of tissue regeneration.

In a preferred embodiment of the invention, the anchors comprise a biocompatible material. In some embodiments, the anchors comprise a biocompatible and biodegradable material.

Thus, in some embodiments, the anchors comprise magnesium.

In some embodiments, the anchors comprise nitinol.

In some embodiments, the anchors comprise stainless steel.

In some embodiments, the anchors comprise a cobalt-chrome nickel alloy.

In some embodiments of the invention, the noted metal anchors include an immunomodulating compound.

In some embodiments, the immunomodulating compound comprises a polysaccharide, including, without limitation, GAGs, dextrans, alginate and chitosan.

In some embodiments, immunomodulating compound comprises a polymeric material, including, without limitation, high molecular weight hyaluronic acid (HMW-HA).

In some embodiments of the invention, the anchors comprise a cross-linked ECM material.

In some embodiments of the invention, the anchors comprise a biocompatible polymeric material selected from the group comprising, without limitation, polyhydroxyalkonates (PHAs), polylactides (PLLA) and polyglycolides (PLGA) and their copolymers, for example poly(ε-caprolactone-co-glycolide)(PCL), polyanhydrides, and like polymers.

In some embodiments of the invention, the anchors comprise an ECM-mimicking biomaterial composition.

In some embodiments of the invention, the ECM-mimicking biomaterial composition comprises poly(glycerol sebacate)(PGS).

In some embodiments of the invention, the ECM-mimicking biomaterial composition comprises PGS and PCL.

In some embodiments of the invention, the anchors comprise expandable members that are similarly capable of transitioning from a first pre-deployment configuration, wherein the pre-deployment configuration of the ECM member is facilitated (or provided), to a post-deployment configuration, wherein the ECM member is supported and positioned proximate the wall of a respiratory tract structure (i.e. host tissue thereof).

In some embodiments of the invention, the anchoring mechanism comprises a microneedle anchoring member having a plurality of biodegradable microneedles or barbs that are adapted to maintain contact of the ECM member to a respiratory tract structure; particularly, the trachea, when disposed therein.

In some embodiments, the microneedle anchoring member comprises a biodegradable polymeric material, an ECM material or a pharmacological or active agent (i.e. drug), e.g., Heparin®, Plavix®, etc., or a combination thereof.

In some embodiments, the microneedle anchoring member comprises a biocompatible and bioabsorbable metal, such as magnesium.

In some embodiments, the microneedles comprise drug-eluting members, which facilitate the direct administration of a pharmacological agent or composition to tissue, e.g. host tissue of a vascular structure.

According to the invention, upon deployment of an regenerative respiratory tract prosthesis of the invention in a respiratory tract structure, the ECM member, i.e. material, covers the target intima surface of the structure, e.g., damaged or diseased region of a trachea, and provides a smooth, non-thrombogenic surface. The ECM material will also induce host tissue proliferation, bioremodeling, including neovascularization, e.g., vasculogenesis, angiogenesis, and intussusception, and regeneration of tissue structures with site-specific structural and functional properties.

In some embodiments of the invention, wherein the ECM member includes a pharmacological agent or the anchoring mechanism comprises a microneedle anchoring member having a plurality of drug-eluting microneedles, a desired biological and/or therapeutic action is also effectuated.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of the upper and lower respiratory tracts of a mammal;

FIG. 2 is a partial section view of a trachea;

FIG. 3 is a perspective view of one embodiment of an ECM tubular member, in accordance with the invention;

FIG. 4 is a perspective view of one embodiment of an ECM trachea prosthesis having two external anchors, in accordance with the invention;

FIG. 5 is a perspective view of another embodiment of an ECM trachea prosthesis having three external anchors, in accordance with the invention;

FIG. 6 is a perspective view of one embodiment of an ECM trachea prosthesis having two internal anchors, in accordance with the invention;

FIG. 7 is a perspective view of another embodiment of an ECM trachea prosthesis having three internal anchors, in accordance with the invention;

FIG. 8 is a end view of the trachea prosthesis shown in FIG. 5 positioned in a trachea, in accordance with the invention;

FIG. 9 is a side plan view of the trachea prosthesis shown in FIG. 5 positioned in a trachea, in accordance with the invention;

FIG. 10A is a front plan view of one embodiment of a single-ring expandable anchor, in accordance with the invention;

FIG. 10B is a side plan view of the single-ring anchor shown in FIG. 10A in a pre-deployment configuration, in accordance with the invention;

FIG. 10C is a perspective view of the single-ring anchor shown in FIG. 10A in a pre-deployment configuration, in accordance with the invention;

FIG. 11 is a perspective view of one embodiment of an ECM trachea prosthesis having two expandable internal anchors, in accordance with the invention;

FIG. 12 is a partial section side plan view of the trachea prosthesis shown in FIG. 11, in accordance with the invention;

FIG. 13 is an end view of the trachea prosthesis shown in FIG. 11, in accordance with the invention;

FIG. 14A is a perspective view of one embodiment of a dual-ring expandable anchor, in accordance with the invention;

FIG. 14B is a perspective view of the dual-ring anchor shown in FIG. 14A in a pre-deployment configuration, in accordance with the invention;

FIG. 14C is a side plan view of the dual-ring anchor shown in FIG. 14A in a pre-deployment configuration, in accordance with the invention;

FIG. 15A is a side plan view of one embodiment of a multi-ring expandable anchor, in accordance with the invention;

FIG. 15B is a perspective view of the multi-ring anchor shown in FIG. 15A in a pre-deployment configuration, in accordance with the invention;

FIG. 15C is a side plan view of the multi-ring anchor shown in FIG. 15A in a pre-deployment configuration, in accordance with the invention;

FIG. 16 is a side plan, sectional view of one embodiment of a trachea prosthesis having the multi-ring anchor shown in FIG. 15A in a post-deployment configuration in a trachea, in accordance with the invention;

FIG. 17 is a perspective view of one embodiment of a trachea prosthesis having the single-ring anchor shown in FIG. 10A and a microneedle member, in accordance with the invention;

FIG. 18 is a side plan, partial sectional view of the trachea prosthesis shown in FIG. 17, in accordance with the invention;

FIG. 19 is a end view of the trachea prosthesis shown in FIG. 17, in accordance with the invention; and

FIG. 20 is a side plan, sectional view of the trachea prosthesis shown in FIG. 17 in a post-deployment configuration in a trachea, in accordance with the invention.

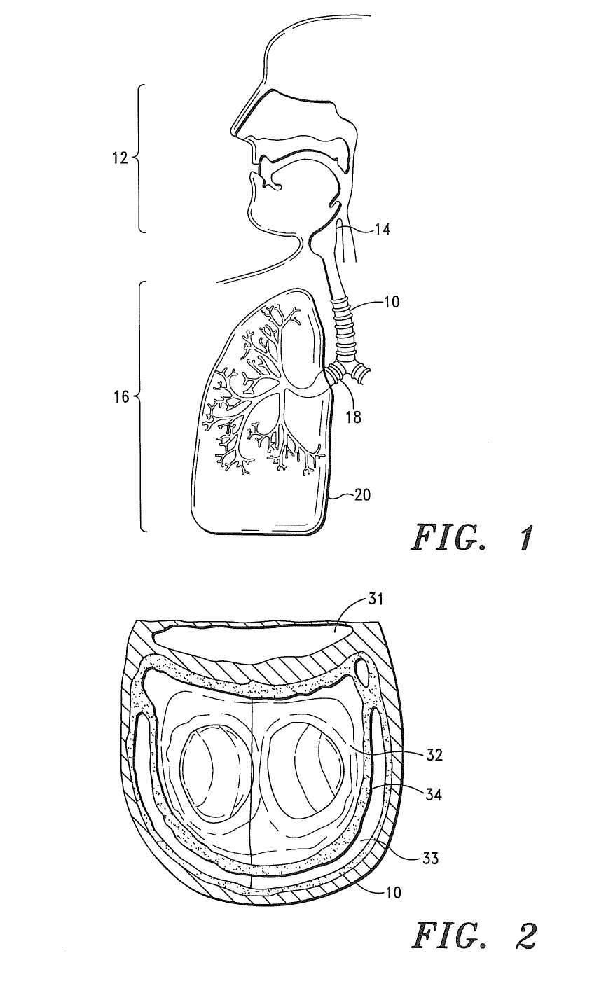

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an active” includes two or more such actives and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

It is also understood that, although the disclosure is primarily directed to treating and/or repairing damage, diseased and/or missing tracheal tissue and segments, the invention is not limited solely to such application. Indeed, the regenerative respiratory tract prostheses and associated methods can readily be employed to treat and/or repair damage, diseased and/or missing tissue and segments of other respiratory tract structure, e.g., bronchi.

### DEFINITIONS

The term “anchor”, as used herein, means and includes a temporary structure that is configured and employed to “temporarily” position a “tracheal prosthesis” proximate the tissue of a trachea or bronchi. As discussed in detail herein, in some embodiments of the invention, the “anchor” is designed and configured to temporary position the ECM prosthesis proximate a recipient's tissue for a predetermined period of time, which, in some embodiments, is preferably within the process of new tissue regeneration.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, ornomentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from epithelium of mesodermal origin, i.e. mesothelial tissue.

The ECM material can additionally be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The term “ECM-mimicking”, as used herein, means and includes a biocompatible and biodegradable biomaterial that induces neovascularization and bioremodeling of tissue in vivo, i.e. when disposed proximate damaged biological tissue. The term “ECM-mimicking” thus includes, without limitation, ECM-mimicking polymeric biomaterials; specifically, poly(glycerol sebacate) (PGS).

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition”, as used herein, thus mean and include a hemostatic agent or composition, including, without limitation, thrombin, fibrinogen, fibronectin, plasminogen, aprotinin, α-2-antiplasmin, α-2 macroglobulin, α-1-antitrypsin, epsilon-aminocaproic acid, tranexamic acid, and a plasmin activator inhibitor, e.g., PAI-1 and PAI-2.

The terms “biologically active agent” and “biologically active composition” also mean and include, without limitation, the following growth factors: platelet derived growth factor (PDGF), epidermal growth factor (EGF), transforming growth factor α (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antiobiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “pharmacological agent”, “active agent”, “drug” and “active agent formulation” can further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and ifosfamide. Chemotherapy agents can also include, without limitation, antimetabolites, such as purine analogues, pyrimidine analogues and antifolates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimuunab, muromonab-CD3, natalizumab, ofatumumab, omalizumab, palivizumab, panitumumab, ranibizumab, rituximab, tocilizumab (atlizumab), tositumomab and trastuzumab.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, a amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “therapeutically effective”, as used herein, means that the amount of the “pharmacological agent” and/or “biologically active agent” and/or “pharmacological composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “abate”, “prevent” and “preventing” are used interchangeably herein, and mean and include reducing the frequency or severity of a condition or disorder. The term does not require an absolute preclusion of the condition or disorder.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder.

The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder.

The term “supplemental” and variations of the term, such as “augmented”, as used in connection with a “biologically active agent or composition”, means and includes the introduction of an additional non-native agent to the bioremodeable or ECM material (and/or an ECM member formed therefrom), and introducing additional native agents to the bioremodelable or ECM material (and/or an ECM member formed therefrom), e.g., fibroblast growth factor-2 (FGF-2) to increase the population of a native agent.

The term “comprise” and variations of the term, such as “comprising” and “comprises,” means “including, but not limited to” and is not intended to exclude, for example, other additives, components, integers or steps.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As discussed above, the present invention is directed to regenerative respiratory tract prostheses for treating and/or repairing damaged, diseased and/or missing respiratory tract structure tissue and/or regions; particularly, tracheal and/or bronchial segments. As will readily be appreciated by one having ordinary skill in the art, the present invention substantially reduces or eliminates the disadvantages and drawbacks associated with prior art methods and apparatus for treating damaged, diseased and/or missing respiratory tract structure segments.

As discussed in detail herein, in a preferred embodiment, the regenerative respiratory tract prostheses of the invention comprise a tubular shaped member comprising an ECM material (hereinafter referred to as “ECM members”). In a preferred embodiment, the ECM members are capable of transitioning from a pre-deployment configuration, wherein the ECM member is capable of being positioned in the respiratory tract, e.g., trachea, to a post-deployment configuration, wherein the ECM member is disposed proximate host tissue of a respiratory tract structure.

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof.

The ECM material can also comprise collagen from mammalian sources.

As is well known in the art, the urinary bladder submucosa comprises an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS).

Other tissues, such as the liver and pancreas have extracellular matrix called basement membrane. Basement membrane generally does not demonstrate the kind of tensile strength found in submucosa. However, other useful properties may be opportunistically employed from the extracellular matrices of such tissues as the liver, pancreas, placenta and lung tissues; all of which have either basement membrane for extracellular matrix or interstitial membrane (as with the lung). For example, the pancreatic extracellular membrane supports beta islet cells that are critical to pancreatic function. Also, for example, the liver is one tissue known to be able to regenerate itself and therefore special qualities may be present in the liver basement membrane that help facilitate that process.

According to the invention, matrices can be used in whole or in part, so that, for example, an extracellular matrix can contain just the basement membrane (or transitional epithelial layer) with the subadjacent tunica propria, the tunica submucosa, tunica muscularis, and tunica serosa. The extracellular matrix component of the composition can contain any or all of these layers, and thus could conceivably contain only the basement membrane portion, excluding the submucosa. However, generally, and especially since the submucosa is thought to contain and support the active growth factors, cytokines and other proteins necessary for in vivo tissue regeneration, the matrix composition from any given source will contain the active extracellular matrix portions that support cell development and differentiation and tissue regeneration.

In some embodiments of the invention, the ECM material comprises an acellular, sterilized ECM material. According to the invention, the ECM material can be sterilized and decellularized by various conventional means. In a preferred embodiment of the invention, the ECM material is sterilized and decellularized via a sterilization and decellularization process disclosed in Co-Pending U.S. PCT Application No. PCT/US12/039413 and Co-Pending U.S. application Ser. No. 13/480,140; which are incorporated by reference herein in their entirety.

As set forth in Co-Pending U.S. application Ser. No. 13/480,140, ECM sterilized via the disclosed process comprises less than 4% DNA content and a dry weight bFGF content of at least 140 pg/mg of the base ECM material. The ECM material also exhibits at least 96% decellurization and a tensile strength of at least 9 N.

As stated above, in some embodiments of the invention, the ECM material (and/or ECM member formed therefrom) comprises at least one supplemental biologically active agent (or composition).

In some embodiments of the invention, the biologically active agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. In particular, Applicant has found that when a statin is added to ECM (wherein a statin augmented ECM composition is formed) and the statin augmented ECM composition is administered to damaged tissue, the statin interacts with the cells recruited by the ECM, wherein the statin augmented ECM composition modulates inflammation of the damaged tissue by modulating several significant inflammatory processes, including restricting expression of monocyte chemoattractant protein-1 (MCP-1) and chemokine (C—C) motif ligand 2 (CCR2).

The properties and beneficial actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

In some embodiments of the invention, the biologically active agent comprises a cell selected from the group comprising human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some embodiments of the invention, the biologically active agent comprises a growth factor selected from the group comprising a platelet derived growth factor (PDGF), epidermal growth factor (EGF), transforming growth factor-α (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor α (TNA-α), and placental growth factor (PLGF).

In some embodiments, the biologically active agent comprises a pharmacological agent (or composition), i.e. an agent or composition that is capable of producing a desired biological effect in vivo, e.g., stimulation or suppression of apoptosis, stimulation or suppression of an immune response, etc.

According to the invention, the pharmacological agent or composition can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

According to the invention, upon deployment of a regenerative respiratory tract prosthesis of the invention in a respiratory tract structure, the ECM member, i.e. material, covers the target intima surface of the structure, e.g., damaged or diseased region of a trachea, and provides a smooth, non-thrombogenic surface. The ECM material will also induce modulated healing, including host tissue proliferation, bioremodeling, including neovascularization, e.g., vasculogenesis, angiogenesis, and regeneration of tissue structures with site-specific structural and functional properties.

In some embodiments of the invention, wherein the ECM member includes a pharmacological composition or, as discussed in detail below, the anchoring mechanism comprises a microneedle anchoring member having a plurality of drug-eluting microneedles, a desired biological and/or therapeutic action is also effectuated.

As also discussed in detail below, in a preferred embodiment of the invention, the regenerative respiratory tract prostheses of the invention include at least one anchoring mechanism. In some embodiments of the invention, the anchoring mechanism comprises a proximal anchor. In some embodiments, the anchoring mechanism comprises a distal anchor. In some embodiments, the anchoring mechanism comprises proximal, distal and mid-region anchors.

In a preferred embodiment, the proximal anchor is disposed proximate the proximal end of the ECM member, the distal anchor is disposed proximate the distal end of the ECM member, and the mid-region anchor is disposed between the proximal and distal anchors.

According to the invention, the regenerative respiratory tract prostheses of the invention can also include a plurality of proximal, distal and/or mid-region anchors. The anchors can also be positioned or spaced in and/or on the ECM member to correspond and facilitate engagement to respiratory tract structure configurations, e.g., natural spacing of tracheal cartilage 33 (see FIG. 2).

According to the invention, the anchors can also comprise various shapes to correspond to various respiratory tract structures. For example, in some embodiments, the anchors have a linear region and a curved region that corresponds to the natural shape of a trachea lumen.

In a preferred embodiment, each anchor comprises a flexible member that substantially conforms to the shape of a respiratory tract structure, such as a trachea, when the anchor(s) and, hence, ECM member engaged thereto, are deployed in the structure. The flexure of the anchors also facilitates the dynamic nature of the trachea 10 and other associated structures, such as the esophagus 31.

In some embodiments of the invention, the anchors of the invention comprise expandable anchors.

In a preferred embodiment, the expandable anchors are similarly configured to conform to the shape of a respiratory tract structure, such as a trachea, when the anchor(s) and, hence, ECM member engaged thereto, are deployed in the structure.

As defined above and discussed in detail below, the term “anchor”, as used herein, means and includes a structure that is configured and employed to temporarily position a structure or member, e.g. an ECM member of the invention, proximate host tissue of a respiratory tract structure. The function of an “anchor” of the invention is thus to temporarily support and position an ECM member of the invention proximate host tissue of a respiratory tract structure.

Thus, in a preferred embodiment of the invention, the anchors of the invention are configured to temporarily support and position an ECM member and, hence, regenerative respiratory tract prostheses formed therefrom, proximate host tissue of a respiratory tract structure, and maintain contact therewith throughout the four phases of the ventilatory cycle, i.e. trigger phase, flow delivery phase, cycle phase and expiratory phase, for a predetermined anchor support period of time within the process of tissue regeneration.

In some embodiments of the invention, wherein a regenerative respiratory tract prosthesis is deployed in a trachea, the anchors thus merely position the ECM member and, hence, prosthesis, proximate the trachea long enough to initiate blood vessel growth. Once blood vessels begin to grow into the ECM member and stem cells attach to the surface, an endothelium layer or lining grows across the ECM member and starts to remodel into healthy, native cells and, thereby, a remodeled, natural trachea structure.

Applicants have found that, in most instances, the ECM member will be completely enclosed in an endothelial lining within a time duration of approximately 3-8 weeks to approximately 2-6 months.

Thus, in a preferred embodiment of the invention, after tissue remodeling commences (i.e. blood vessel growth is initiated), the function of the anchors transitions from a positioning and supporting function, wherein the anchors position and support the ECM member proximate the host tissue of the respiratory tract structure, to a reinforcing function, wherein the anchors merely reinforce the ECM member and/or remodeled tissue during (and after) the tissue regeneration process.

Depending on the material employed to form the anchors, the anchors could be completely absorbed or remain in place with no continued functional use, i.e. function similar to rebar in a matrix.

As stated above, in some embodiments, the anchoring mechanism comprises expandable anchors. In the noted embodiments, the expandable proximal anchor is similarly disposed proximate the proximal end of the ECM member, the expandable distal anchor is disposed proximate the distal end of the ECM member, and the mid-region anchor is disposed between the expandable proximal and distal anchors.

In a preferred embodiment, the expandable anchors are also capable of transitioning from a pre-deployment configuration, wherein the pre-deployment configuration of the ECM member is facilitated (or provided), to a post-deployment configuration, wherein the ECM member is supported and positioned proximate a respiratory tract structure, e.g., trachea for a predetermined temporary anchor support period of time.

In some embodiments of the invention, the anchors preferably comprise a biocompatible material. In some embodiments, the anchors preferably comprise a biocompatible and biodegradable material.

Thus, in some embodiments, the anchors comprise magnesium.

In some embodiments, the anchors comprise nitinol.

In some embodiments, the anchors comprise stainless steel.

In some embodiments, the anchors comprise a cobalt-chrome nickel alloy.

In some embodiments of the invention, the noted metal anchors include a coating of an immunomodulating compound that suppresses acute immune responses, while up regulating chronic immune response (i.e. tissue reconstruction).

In some embodiments, the immunomodulating compound comprises a polysaccharide, including, without limitation, GAGs, dextrans, alginate and chitosan.

In some embodiments, immunomodulating compound comprises a polymeric material, including, without limitation, high molecular weight hyaluronic acid (HMW-HA).

In some embodiments, the anchors comprise a biocompatible polymeric material selected from the group comprising, without limitation, polyhydroxyalkonates (PHAs), polylactides (PLLA) and polyglycolides (PLGA) and their copolymers, for example poly(ε-caprolactone-co-glycolide), polyanhydrides, and like polymers.

In some embodiments, the support member comprises Artelon™.

In some embodiments of the invention, the anchors compri^{se} artificial/semi-synthetic hyaline cartilage.

In some embodiments of the invention, the anchors compr^{is}e a collagen matrix having chondroitin-6-sulfate.

In some embodiments of the invention, the anchors comp^{ri}se a cross-linked ECM material.

In some embodiments of the invention, the anchors com^{pr}ise an ECM-mimicking biomaterial composition.

In some embodiments of the invention, the ECM-mimickⁱⁿg biomaterial composition comprises poly(glycerol sebacate)(PGS).

Applicant has found that PGS exhibits numerous bene^{fi}cial properties that provide several beneficial biochemical actions or activities. The properties and beneficial actions resulting therefrom are discussed in detail below.

### PGS Physical Properties

PGS is a condensate of the non-immunogenic compositions glycerol (a simple sugar alcohol) and sebacic acid (a naturally occurring dicarboxylic acid), wherein, glycerol and sebacic acid are readily metabolized when proximate mammalian tissue. The non-immunogenic properties substantially limit the acute inflammatory responses typically associated with other “biocompatible” polymers, such as ePTFE (polytetrafluoroethylene), that are detrimental to bioremodeling and tissue regeneration.

The mechanical properties of PGS are substantially similar to that of biological tissue, wherein, the value of the Young's modulus of PGS is between that of a ligament (in KPa range) and tendon (in GPa range). The strain to failure of PGS is also similar to that of arteries and veins (i.e. over 260% elongation).

The tensile strength of the PGS is at least 0.28±0.004 MPa. The Young's modulus and elongation are at least 0.122±0.0003 and at least 237.8±0.64%, respectively. For applications requiring stronger mechanical properties and a slower biodegradation rate, PGS can be blended with PCL, i.e. a biodegradable elastomer.

### ECM Mimicking Properties/Actions

It has been found that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

In some embodiments of the invention, the ECM-mimicking biomaterial composition comprises PGS and PCL.

In some embodiments of the invention, the anchoring mechanism comprises (or includes) a microneedle anchoring member having a plurality of biodegradable microneedles or barbs that are configured to maintain contact of the ECM prostheses proximate the trachea, i.e. tissue thereof, when disposed therein.

In some embodiments, the microneedles comprise drug-eluting members that facilitate the direct administration of a pharmacological agent to host tissue, e.g. host tissue of a vascular structure.

In a preferred embodiment of the invention, upon deployment of a regenerative respiratory tract prosthesis of the invention to damaged or diseased biological tissue, the prosthesis induces ‘modulated healing” of the biological tissue.

The term “modulated healing”, as used herein, and variants of this language means and includes modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the regenerative respiratory tract prostheses of the invention are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase.

In some embodiments, “modulated healing” refers to the ability of a regenerative respiratory tract prosthesis to alter a substantial inflammatory phase (e.g., platelet or fibrin deposition) at the beginning of the tissue healing process. As used herein, the phrase “alter a substantial inflammatory phase” refers to the ability of an ECM construct to substantially reduce the inflammatory response at an injury site.

In such an instance, a minor amount of inflammation may ensue in response to tissue injury, but this level of inflammation response, e.g., platelet and/or fibrin deposition, is substantially reduced when compared to inflammation that takes place in the absence of a regenerative respiratory tract prosthesis of the invention.

For example, the a regenerative respiratory tract prostheses discussed herein have been shown experimentally to delay or alter the inflammatory response associated with damaged tissue, as well as excessive formation of connective fibrous tissue following tissue damage or injury. The regenerative respiratory tract prostheses have also been shown experimentally to delay or reduce fibrin deposition and platelet attachment to a blood contact surface following tissue damage.

In some embodiments of the invention, “modulated healing” refers to the ability of a regenerative respiratory tract prosthesis of the invention to induce host tissue proliferation, bioremodeling, including neovascularization, e.g., vasculogenesis, angiogenesis, and regeneration of tissue structures with site-specific structural and functional properties.

Accordingly, the regenerative respiratory tract prostheses of the invention provide an excellent means for treating damaged and/or diseased tissue, and/or missing segments of respiratory tract structures.

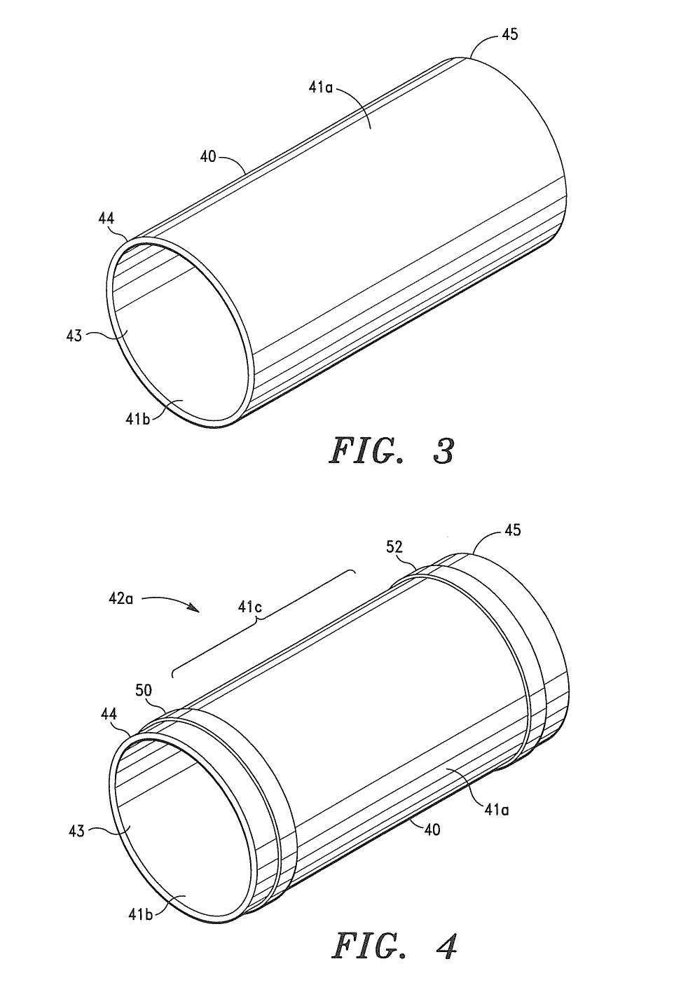

Referring now to FIGS. 3-7, several embodiments of regenerative respiratory tract prostheses of the invention will be described in detail.

As indicated above, the regenerative respiratory tract prostheses of the invention comprise a tubular shaped member having a lumen therethrough. As also indicated above, in a preferred embodiment, the tubular members comprise expandable ECM members that are capable of transitioning from a pre-deployment configuration, wherein the ECM member is capable of being positioned in a respiratory tract structure, to a post-deployment configuration, wherein the member is disposed proximate host tissue of the respiratory tract structure.

Referring now to FIG. 3, there is shown one embodiment of an expandable ECM member 40 of the invention having a lumen 43 that extends therethrough. In some embodiments of the invention, the ECM member 40 comprises a single layer of ECM material. In some embodiments, the ECM member 40 comprises multiple, preferably wrapped, layers of ECM material.

As stated above, according to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

As set forth above, in a preferred embodiment, the regenerative respiratory tract prostheses of the invention include at least one anchoring mechanism. In some embodiments of the invention, the anchoring mechanism comprises a proximal anchor. In some embodiments, the anchoring mechanism comprises a distal anchor. In some embodiments, the anchoring mechanism comprises proximal and distal anchors. In some embodiments, the anchoring mechanism comprises proximal, distal and mid-region anchors.

Referring now to FIG. 4, there is shown one embodiment of a regenerative respiratory tract prosthesis 42a having an ECM member 40, and proximal 50 and distal 52 anchors. As illustrated in FIG. 4, the proximal 50 and distal 52 anchors are disposed proximate the outer surface 41a of the ECM member 40 and proximate the proximal 44 and distal 45 ends thereof.

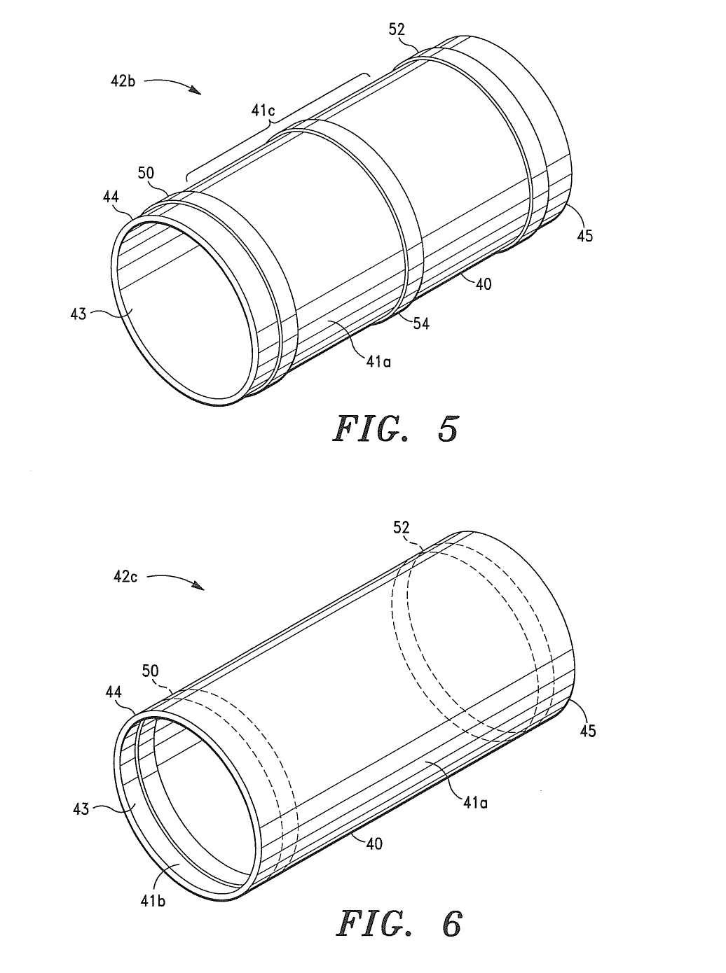

Referring now to FIG. 5, there is shown another embodiment of a regenerative respiratory tract prosthesis 42b that similarly comprises an ECM member 40, and proximal 50 and distal 52 anchors. In the illustrated embodiment, the trachea prosthesis 42b further includes a mid-region anchor 54.

As illustrated in FIG. 5, the proximal 50 and distal 52 anchors are similarly disposed proximate the outer surface 41a of the ECM member 40 and proximate the proximal 44 and distal 45 ends thereof. The mid-region anchor 54 is disposed proximate the mid-region 41c of the ECM member 40.

According to the invention, the mid-region anchor 54 can be positioned at any desired ECM member region between the proximal 50 and distal 52 anchors.

As indicated above, the prosthesis 42 can also include a plurality of proximal 50 and/or distal 52 and/or mid-region anchors. The anchors 50, 52, 54 can be positioned or spaced on (or in) the ECM member 40 to correspond and facilitate engagement to various respiratory structure configurations and/or features, e.g. spacing of tracheal cartilage 33.

According to the invention, the proximal 50, distal 52 and mid-region 54 anchors can also be disposed in the ECM member lumen 43; preferably, proximate the inner surface 41b of the lumen 43.

Referring now to FIG. 6, there is shown one embodiment of a regenerative respiratory tract prosthesis 42c having proximal 50 and distal 52 anchors disposed proximate the inner surface 41b of the ECM member 40 and proximate the proximal 44 and distal 45 ends thereof.

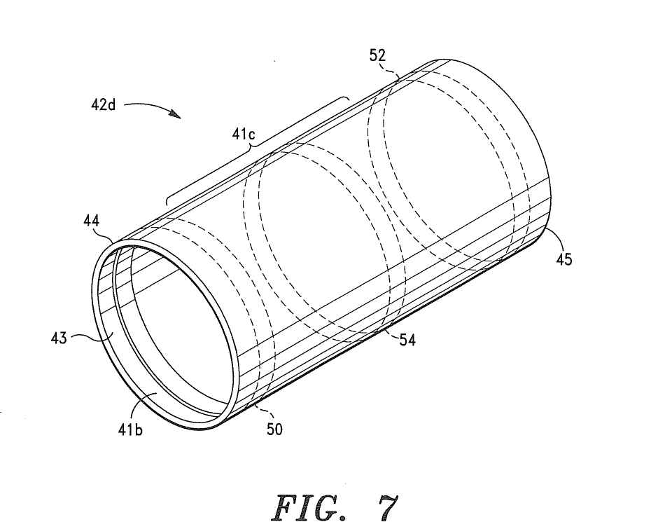

Referring now to FIG. 7, there is shown one embodiment of a regenerative respiratory tract prosthesis 42d having proximal 50 and distal 52 anchors disposed proximate the inner surface 41b of the ECM member 40 and proximate the proximal 44 and distal 45 ends thereof, and a mid-region anchor 54 that is disposed in the ECM member lumen 43 proximate the inner surface 41b thereof.

According to the invention, the anchors 50, 52, 54 can be secured to the outer 41a and/or inner surface 41b by various conventional means. In a preferred embodiment, the anchors 50, 52 54 are sutured to the outer 41a and/or inner surface 41b.

According to the invention, the anchors 50, 52, 54 can comprise various configurations and materials. In a preferred embodiment, the anchors 50, 52, 54 comprise substantially circular or single ring members.

In some embodiments of the invention, the ring anchors have a substantially circular cross-section. In some embodiments, the ring anchors have a planar cross-section, as illustrated in FIGS. 4 and 5.

According to the invention, the planar shaped anchors can comprise various widths. In a preferred embodiment, the width of the planar shaped anchors is in the range of approximately 0.25 mm to 1.0 cm or greater.

In some embodiments of the invention, the ring anchors preferably have a thickness in the range of approximately 0.25-1.0 mm. In some embodiments, the anchors preferably have a thickness in the range of approximately 0.05-0.25 mm.

In some embodiments of the invention, the anchors 50, 52, 54 of the invention have an elastic modulus in the range of approximately 0.01-5.0 GPA, more preferably, in the range of approximately 0.1-2.5 GPA

As stated above, according to the invention, the anchors 50, 52, 54 can comprise various biocompatible materials.

In some embodiments, the anchors 50, 52, 54 thus comprise magnesium.

In some embodiments, the anchors 50, 52, 54 comprise stainless steel.

In some embodiments of the invention, the anchors 50, 52, 54 comprise a biocompatible shape memory alloy, including, without limitation, Nitinol®.

In some embodiments, the anchors 50, 52, 54 comprise a cobalt-chrome nickel alloy.

As also stated above, in some embodiments of the invention, the anchors 50, 52, 54 include a coating of an immunomodulating compound that suppresses acute immune responses, while up-regulating chronic immune response (i.e. tissue reconstruction).

In some embodiments, the immunomodulating compound comprises a polysaccharide, including, without limitation, GAGs, dextrans, alginate and chitosan.

In some embodiments, immunomodulating compound comprises a polymeric material, including, without limitation, high molecular weight hyaluronic acid (HMW-HA).

In some embodiments of the invention, the anchors 50, 52, 54 comprise a polymeric material, more preferably, a biocompatible and biodegradable polymeric material, such as, without limitation, polyesters, poly(amino acids), polyanhydrides, polyorthoesters, polyurethanes, polycarbonates, homopolymers and copolymers of poly(lactic acid) and poly(glycolic acid), copolyesters of e-caprolactone, trimethylene carbonate, and para-dioxanone, and like polymeric materials.

In a preferred embodiment, the anchors 50, 52, 54 comprise a biocompatible polymeric material selected from the group comprising, without limitation, polyhydroxyalkonates (PHAs), polylactides (PLLA) and polyglycolides (PLGA) and their copolymers, for example poly(ε-caprolactone-co-glycolide), polyanhydrides, and like polymers.

In some embodiments of the invention, the anchors 50, 52, 54 comprise artificial/semi-synthetic hyaline cartilage.

In some embodiments, the anchors 50, 52, 54 comprise a collagen matrix having chondroitin-6-sulfate.

In some embodiments, the anchors 50, 52, 54 comprise a cross-linked ECM material.

In some embodiments of the invention, the anchors 50, 52, 54 comprise an ECM-mimicking biomaterial composition.

In some embodiments of the invention, the ECM-mimicking biomaterial composition comprises PGS.

In some embodiments of the invention, the ECM-mimicking biomaterial composition comprises PGS and PCL.

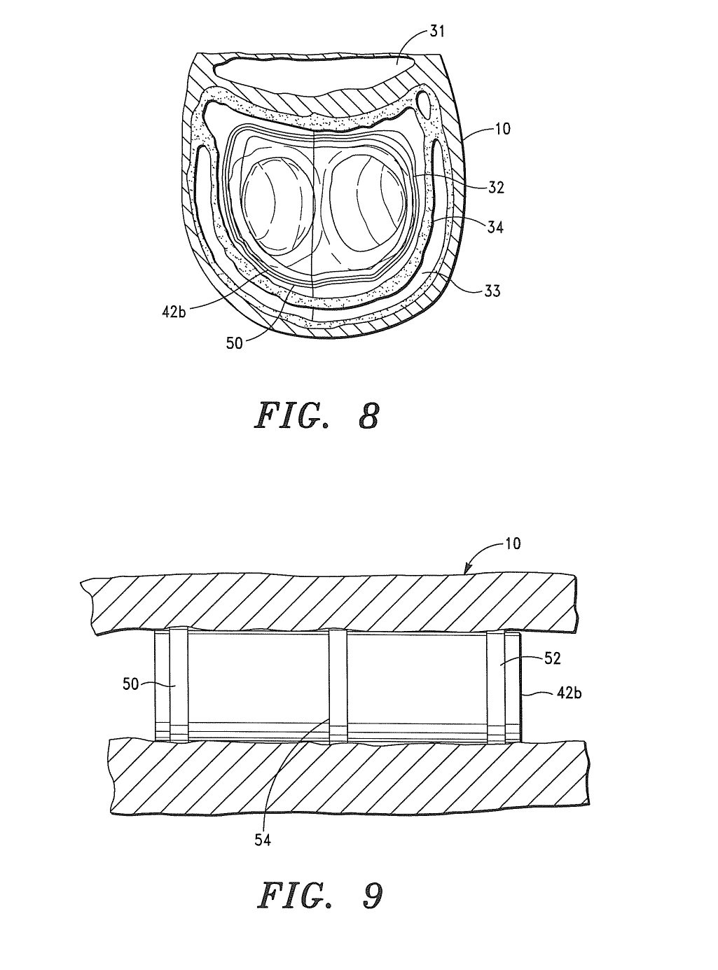

Referring now to FIGS. 8 and 9, there is shown regenerative respiratory tract prosthesis 42b (shown in FIG. 5) positioned in a trachea 10 of a subject. As stated above, upon deployment of the regenerative prosthesis 42b in the trachea 10, the prosthesis 42b covers the target intima surface of the trachea 10, e.g., damaged or diseased region of the trachea 10, and provides a smooth, non-thrombogenic surface. The ECM material and, hence, prosthesis 42b formed therefrom, will also induce host tissue proliferation, bioremodeling, including neovascularization, e.g., vasculogenesis, angiogenesis, and regeneration of tissue structures with site-specific structural and functional properties.

In some embodiments of the invention, wherein the ECM material includes a pharmacological agent (or the anchoring mechanism comprises a microneedle anchoring member having a plurality of drug-eluting microneedles), a desired biological and/or therapeutic action is also effectuated.

As indicated above, in some embodiments of the invention, the anchoring mechanism comprises at least one, more preferably a plurality of expandable anchors. Suitable expandable anchors are described in detail in U.S. Pat. No. 8,778,012, which is incorporated by reference herein in its entirety.

As indicated above and discussed in detail in the '012 patent, the expandable anchors are similarly capable of transitioning from a pre-deployment configuration, wherein a pre-deployment configuration of the ECM member is facilitated (or provided), to a post-deployment configuration, wherein the ECM member is supported and positioned proximate a respiratory tract structure for an anchor support period of time.

In some embodiments of the invention, the expandable anchors of the invention completely degrade after the anchor support period of time.

In some embodiments of the invention, degradation of the expandable anchors is controlled, whereby substantially all of the anchor material is absorbed proximate the ECM member 40. According to the invention, the noted controlled degradation is achieved by the defined ring thicknesses and supporting forces exerted on the ECM member 40 by the anchors, and the remodeling characteristics effectuated by the ECM member 40 (or material thereof), whereby, when a regenerative respiratory tract prosthesis of the invention is deployed in a respiratory tract structure, i.e. the ECM member 40 and anchors are in a post-deployment configuration, new tissue is generated and encases the anchors. The anchor material is then absorbed while encased by the new tissue, which substantially reduces or eliminates the possibility of the anchor fragments flowing into the respiratory tract.

In some embodiments of the invention, controlled degradation is achieved via encasement of the anchor(s) in an endothelial lining after tissue remodeling commences for a defined period of time during and post healing, which similarly substantially reduces or eliminates the possibility of the anchor fragments flowing into the respiratory tract.

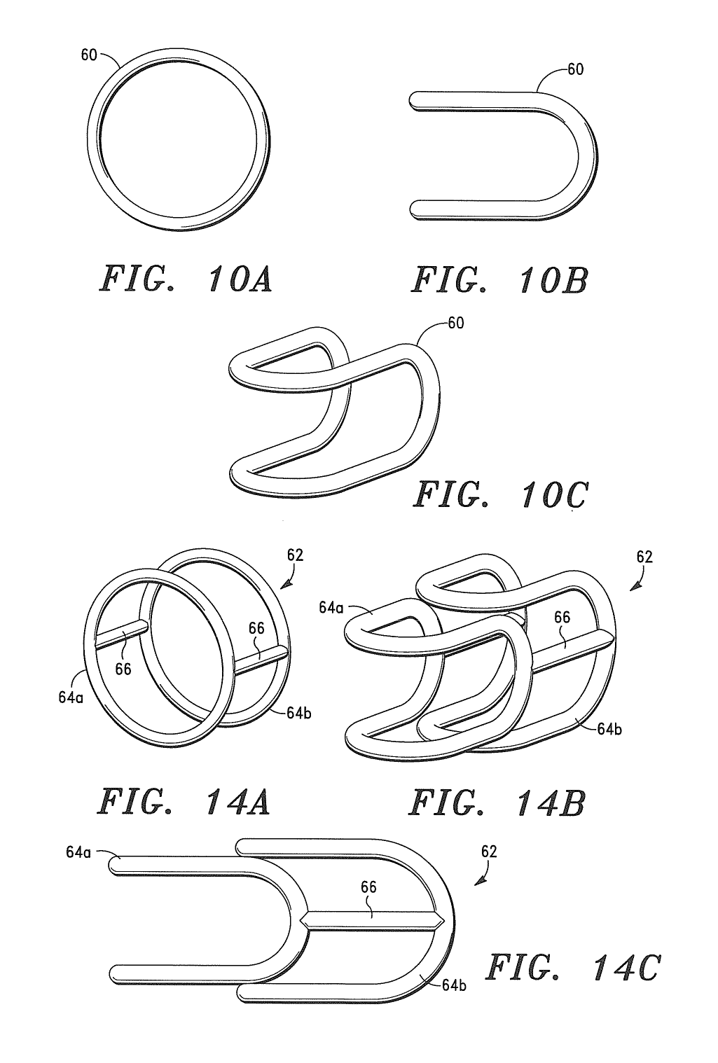

Referring now to FIG. 10A-10C, there is shown one embodiment of an expandable anchor 60 of the invention.

As illustrated in FIG. 10A (and shown in FIG. 11), the expandable anchor 60 preferably has a substantially circular “post-deployment” configuration or shape, i.e. shape after placement in an upper respiratory tract structure, such as a trachea. To facilitate deployment of a regenerative respiratory tract prosthesis of the invention into a respiratory tract structure, the expandable anchor 60 also has a predetermined initial or “pre-deployment” configuration or shape.

As discussed in detail below, the initial or pre-deployment configuration is dependent upon the anchor material. Thus, in some embodiments of the invention, the anchor 60 is formed with a pre-deployment configuration, such as shown in FIGS. 10B and 10C.

In some embodiments of the invention, the expandable anchor 60 is formed with a substantially circular shape, such as shown in FIG. 10A. According to the invention, the anchor 60 is then re-configured (via mechanical force means) to achieve the pre-deployment configuration shown in FIGS. 10B and 10C.

According to the invention, the expandable anchor 60 can comprise a biocompatible metal, including, without limitation, stainless steel and magnesium. The metal can also comprise a cobalt-chrome-nickel alloy.

In these embodiments, the expandable anchor 60 is initially formed in a pre-deployment configuration. In a preferred embodiment of the invention, the pre-deployment configuration comprises a substantially saddle shape, such as shown in FIGS. 10B and 10C.

Referring now to FIGS. 11-13, there is shown one embodiment of a regenerative respiratory tract prosthesis of the invention (denoted “42e”) having expandable anchors 60 disposed in the ECM member lumen 43 proximate the inner surface 41b, and proximate the proximal 44 and distal 45 ends of the member 40. In a preferred embodiment, the anchors 60 are engaged to the lumen inner surface 41b.

According to the invention, when the expandable anchors 60 are engaged to the ECM member 40, the pre-deployment configuration shape of the anchors 60 reduces the effective diameter of the ECM member 40, i.e. places the ECM member 40 is a pre-deployment configuration, which allows the ECM member 40 and, hence, prosthesis 42e to be easily placed in a respiratory tract structure.

After the trachea prosthesis is placed at a desired position in a respiratory tract structure, the prosthesis 42e is expanded, e.g. via a balloon, whereby the expandable anchors 60 are plastically deformed (i.e. re-configured or expanded) to achieve a substantially circular (and permanent) shape (see FIGS. 11 and 13), and whereby the anchors 60 temporarily position the ECM member 40, and, hence, prosthesis 42e proximate host tissue of the respiratory tract structure.

In some embodiments of the invention, the anchors or rings 60 comprise a biocompatible shape memory alloy, including, without limitation, Nitinol®. In these embodiments, the anchors 60 are initially formed in a substantially circular pre-deployment configuration or shape and subsequently heat-treated at a first temperature (i.e. shape set heat treatment).

The anchors 60 are then deformed or formed in a pre-deployment configuration or shape. In a preferred embodiment of the invention, the pre-deployment configuration or shape comprises a substantially saddle shape, such as shown in FIGS. 10B and 10C.

After the prosthesis 42e is placed at a desired position in a respiratory tract structure, the anchors 60 transition to an austenitic phase (i.e. the temperature of the anchors 60 reach and exceed the Nitinol® transition temperature by virtue of the body temperature) and recover (or expand to) their original circular shape, whereby the anchors 60 similarly temporarily position the ECM member 40, and, hence, prosthesis 42e proximate host tissue of the respiratory tract structure.

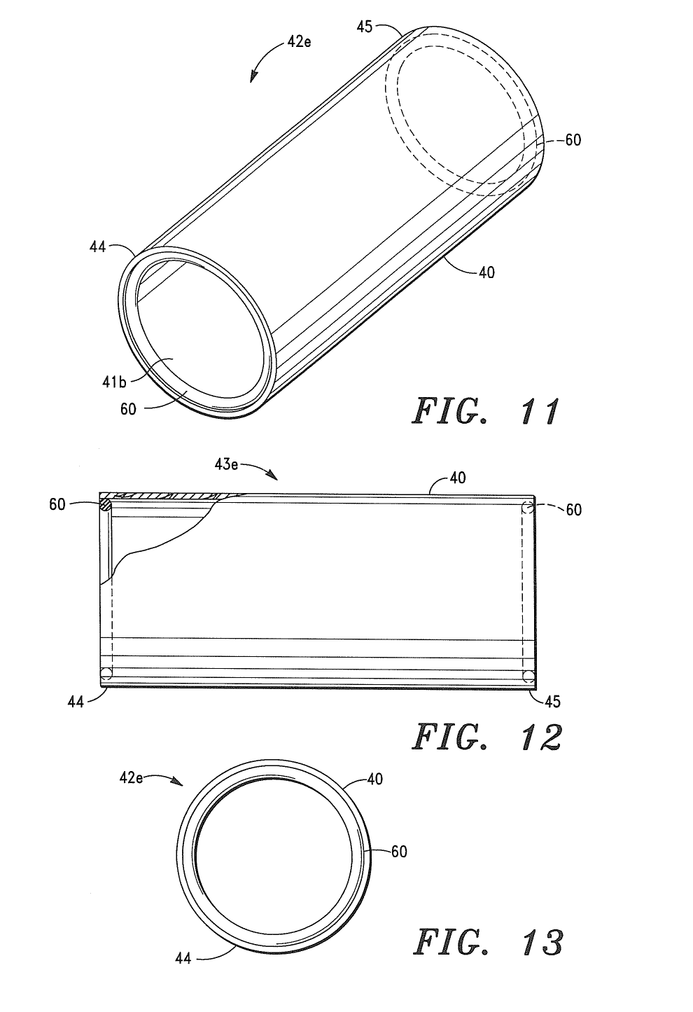

In some embodiments of the invention, the expandable anchors comprise dual-ring members or structures. Referring now to FIGS. 14A-14C, there is shown one embodiment of an expandable dual-ring anchor 62.

As illustrated in FIG. 14A, the anchor 62 includes first and second rings 64a, 64b and at least two connecting links 66. In a preferred embodiment, the rings 64a, 64b are oriented in substantially parallel planes.

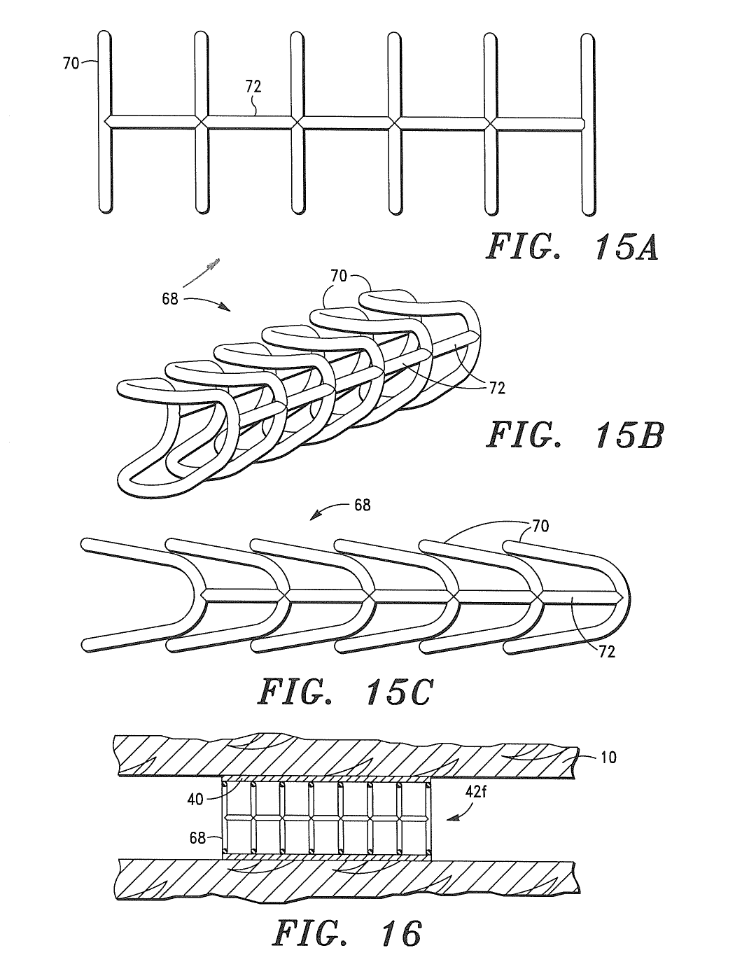

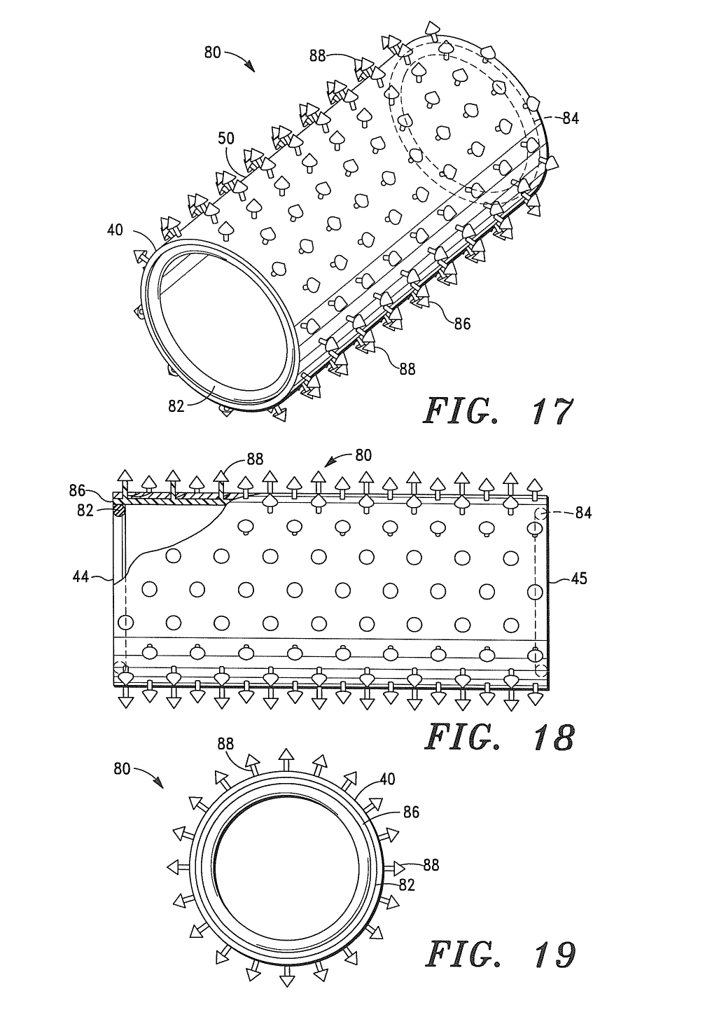

In some embodiments of the invention, the expandable anchors comprise multi-ring members or structures. Referring now to FIGS. 15A-15C, there is shown one embodiment of an expandable multi-ring anchor 68.

As illustrated in FIG. 15A, the multi-ring anchor 68 includes rings 70 and connecting links 72. According to the invention, the anchor 68 can comprise any number of rings 70 and connecting links 72, e.g., 4, 6 or 7 rings 70.

According to the invention, the anchor 68 is similarly capable of transitioning from a pre-deployment configuration, wherein the pre-deployment configuration of the ECM member 40 is facilitated, to a post-deployment configuration, wherein the ECM member 40 is supported and positioned proximate a respiratory tract structure for a predetermined temporary anchor support period of time.

According to the invention, each anchor ring 70 and link 72 can similarly comprise any of the aforementioned materials.

Preferably, each anchor ring 70 similarly has a substantially circular post-deployment configuration.

To facilitate deployment of a regenerative respiratory tract prosthesis of the invention into a respiratory tract structure, each anchor ring 70 similarly has a predetermined initial or pre-deployment configuration or shape. The pre-deployment configuration or shape is similarly dependent upon the material employed to form the rings 70.

Depending upon the anchor ring material, each ring 70 preferably achieves a post-deployment shape in a respiratory tract structure, such as shown in FIG. 16, by the aforementioned means, whereby the anchor 68 temporarily positions the ECM member 40, and, hence, prosthesis 40f proximate host tissue of the respiratory tract structure.

A significant advantage of the multi-ring anchor 30 is that the anchor 30 can support a much longer span inside of the ECM member 40 with optimal support using multiple rings 72 with the same separation distance, or varied for optimal support with multiple anchors, or one anchor the complete length of the ECM member 40, and, hence, prosthesis.

In some embodiments of the invention, the anchoring mechanism includes a microneedle anchoring member having plurality of biodegradable microneedles or barbs that are adapted to maintain contact of the ECM member 40 proximate a respiratory tract structure when disposed therein.

As indicated above, suitable microneedle anchoring members are disclosed in U.S. Pat. No. 8,778,012.

According to the invention, various shaped microneedles or barbs can be employed within the scope of the invention; provided, the microneedle or barb has a head (or head region) that is able to pierce tissue and remain engaged to the tissue for a predetermined period of time.

In some embodiments, the microneedle anchoring member and, hence, microneedles comprise a biodegradable polymeric material, an ECM material or a pharmacological agent or composition (i.e. drug), e.g., Heparin®, Plavix®, etc., or a combination thereof.

In some embodiments, the microneedle anchoring member and, hence, microneedles comprise a biocompatible and bioabsorbable metal, such as magnesium.

In some embodiments, the microneedles comprise drug-eluting members that facilitate the direct administration of a pharmacological agent or composition, preferably, at least one of the aforementioned pharmacological agents or compositions, to host tissue.

In some embodiments of the invention, the drug-eluting capability is facilitated by forming at least one, more preferably, each microneedle out of a pharmaceutical composition, whereby upon engagement of the biodegradable microneedles to a recipient's tissue, the microneedles dissolve or degrade and the pharmaceutical composition is administered to the recipient at the engagement site.

In some embodiments, the drug-eluting capability is facilitated by coating at least one, more preferably, each microneedle with a pharmaceutical composition, whereby upon engagement of the microneedles to a recipient's tissue, the pharmaceutical composition is absorbed and, hence, administered to the recipient.

In some embodiments of the invention, at least one, more preferably, each microneedle has an internal reservoir that is adapted to receive and contain a pharmaceutical composition therein. According to the invention, upon engagement of the biodegradable microneedles to a recipient's tissue, the microneedles dissolve or degrade and the pharmaceutical composition contained in the reservoir is administered to the recipient.

In some embodiments of the invention, the microneedle has an internal reservoir that is adapted to receive and contain a pharmaceutical composition therein and at least one, more preferably, a plurality of lumens in communication with the reservoir and, hence, pharmaceutical composition contained therein. The microneedle also includes a biodegradable or bioabsorbable coating (or sealing layer) on the outer surface to temporarily seal reservoir and inter-connected lumens. Upon engagement of the microneedles to a recipient's tissue, the coating dissolves or degrades and the pharmaceutical composition contained in the reservoir is administered to the recipient via the microneedle lumens.

As set forth in detail in U.S. Pat. No. 8,778,012, the on-set and rate of administration of a pharmacological composition can be determined and regulated by, among other things, the composition and/or properties of the base microneedle, e.g. dissolution rate, size of lumens, etc., and the composition and/or properties of the pharmacological and sealing coatings.

Referring now to FIGS. 17-20, there is shown one embodiment of a regenerative respiratory structure prosthesis 80 of the invention having a tubular ECM member 40, proximal and distal anchors 82, 84, and a microneedle member (or support scaffold) 86. As illustrated in FIG. 17, the support scaffold 86 includes a plurality of microneedles 88, which preferably extend through and project out of the ECM member 40.

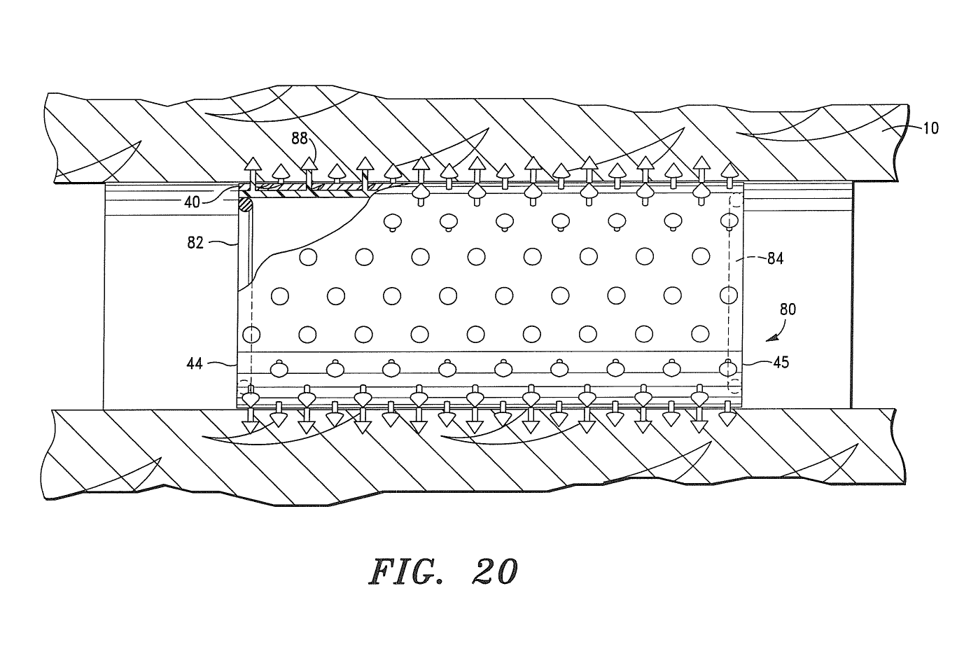

As illustrated in FIG. 20, upon deployment of the prosthesis 80 in a trachea 10 (or other respiratory tract structure), the anchors 82, 84 position and maintain the proximal 44 and distal 45 ends of the ECM member 40 proximate the trachea 10. The microneedles 88 also engage the trachea 10 and secure the ECM member 40 proximate the trachea 10.

As will readily be appreciated by one having ordinary skill in the art, the present invention provides numerous advantages compared to prior art apparatus and methods for treating and/or repairing damaged or diseased respiratory tract structures. Among the advantages are the following:
The provision of regenerative respiratory tract prostheses that can readily be placed in respiratory tract structures.
The provision of regenerative respiratory tract prostheses that substantially reduce or eliminate (i) the harsh biological responses associated with conventional polymeric and metal prostheses, and (ii) the formation of biofilm, inflammation and infection.
The provision of regenerative respiratory tract prostheses that induce host tissue proliferation, bioremodeling and regeneration of new tissue and tissue structures with site-specific structural and functional properties.
The provision of regenerative respiratory tract prostheses that are configured to administer a pharmacological agent to the tissues of upper respiratory tract structures and, thereby produce a desired biological and/or therapeutic effect.
The provision of regenerative respiratory tract prostheses that include unique anchor members that temporarily position the prostheses proximate target tissue of a respiratory tract structure for a pre-determined period of time and have controlled degradation characteristics.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A regenerative respiratory tract prosthesis, comprising:
a tubular member comprising an decellularized extracellular matrix (ECM) material, said tubular member having a interior and exterior surface, and proximal and distal ends, said interior surface defining a conduit through said member;
at least a first biodegradable anchor disposed on said tubular member exterior surface and proximate said tubular member proximal end; and
at least a second biodegradable anchor disposed on said tubular member exterior surface proximate said tubular member distal end,
said first and second anchors comprising a biodegradable polymeric material selected from the group consisting of polyhydroxyalkonates (PHAs), polylactides (PLLA) and polyglycolides (PLGA) and copolymers of said PLGA.

2. The respiratory tract prosthesis of claim 1, wherein said prosthesis further comprises at least a third biodegradable anchor disposed on said tubular member exterior surface between said first and second anchors.

3. The respiratory tract prosthesis of claim 1, wherein said first and second anchors comprise polymeric material selected from the group consisting of polyesters, poly(amino acids), polyanhydrides, polyorthoesters, polyurethanes, polycarbonates, homopolymers and copolymers of poly(lactic acid) and poly(glycolic acid), copolyesters of e-caprolactone, trimethylene carbonate, and para-dioxanone.

4. The respiratory tract prosthesis of claim 1, wherein said first and second anchors comprise artificial/semi-synthetic hyaline cartilage.

5. The respiratory tract prosthesis of claim 1, wherein said first and second anchors comprise a collagen matrix comprising chondroitin-6-sulfate.

6. The respiratory tract prosthesis of claim 1, wherein said first and second anchors comprise magnesium.

7. The respiratory tract prosthesis of claim 1, wherein said first and second anchors comprise cross-linked extracellular matrix (ECM).

8. The respiratory tract prosthesis of claim 1, wherein said ECM material comprises ECM selected from the group consisting of small intestine submucosa (SIS), urinary bladder submucosa (UBS), stomach submucosa (SS), dermal extracellular matrix, subcutaneous extracellular matrix, gastrointestinal extracellular matrix, placental extracellular matrix, omomentum extracellular matrix, cardiac extracellular matrix, kidney extracellular matrix, pancreas extracellular matrix, lung extracellular matrix, and combinations thereof.

9. The respiratory tract prosthesis of claim 1, wherein said ECM material comprises at least one supplemental biologically active agent.

10. The respiratory tract prosthesis of claim 9, wherein said supplemental biologically active agent comprises a HMG-CoA reductase inhibitor selected from the group consisting of atorvastatin, cerivastatin, fluvastatin, lovastatin, mevastatin, pitavastatin, pravastatin, rosuvastatin, and simvastatin.

11. The respiratory tract prosthesis of claim 9, wherein said supplemental biologically active agent comprises a cell selected from the group consisting of human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

12. The respiratory tract prosthesis of claim 9, wherein said supplemental biologically active agent comprises a growth factor selected from the group comprising a platelet derived growth factor (PDGF), epidermal growth factor (EGF), transforming growth factor-α (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor α (TNA-α), and placental growth factor (PLGF).

13. The respiratory tract prosthesis of claim 1, wherein said ECM material comprises at least one pharmacological agent.

14. The respiratory tract prosthesis of claim 13, wherein said pharmacological agent comprises an agent selected from the group consisting of antibiotics, anti-virals, anesthetics, analgesics and anti-inflammatories.

15. A regenerative respiratory tract prosthesis, comprising:
a tubular member comprising an acellular extracellular matrix (ECM) material, said tubular member having a interior and exterior surface, and proximal and distal ends, said interior surface defining a conduit through said member;
at least a first biodegradable anchor disposed on said tubular member interior surface and proximate said tubular member proximal end; and
at least a second biodegradable anchor disposed on said tubular member interior surface proximate said ECM member distal end,
said first and second anchors comprising a biodegradable polymeric material selected from the group consisting of polyhydroxyalkonates (PHAs), polylactides (PLLA) and polyglycolides (PLGA) and copolymers of said PLGA.

16. The respiratory tract prosthesis of claim 15, wherein said prosthesis further comprises at least a third biodegradable anchor disposed on said tubular member interior surface between said first and second anchors.

17. The respiratory tract prosthesis of claim 15, wherein said first and second anchors comprise polymeric material selected from the group consisting of polyesters, poly(amino acids), polyanhydrides, polyorthoesters, polyurethanes, polycarbonates, homopolymers and copolymers of poly(lactic acid) and poly(glycolic acid), copolyesters of e-caprolactone, trimethylene carbonate, and para-dioxanone.

18. The respiratory tract prosthesis of claim 15, wherein said first and second anchors comprise artificial/semi-synthetic hyaline cartilage.

19. The respiratory tract prosthesis of claim 15, wherein said first and second anchors comprise a collagen matrix comprising chondroitin-6-sulfate.

20. The respiratory tract prosthesis of claim 15, wherein said first and second anchors comprise magnesium.

21. The respiratory tract prosthesis of claim 15, wherein said first and second anchors comprise cross-linked extracellular matrix (ECM).

22. The respiratory tract prosthesis of claim 15, wherein said ECM material comprises ECM selected from the group consisting of small intestine submucosa (SIS), urinary bladder submucosa (UBS), stomach submucosa (SS), dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

23. The respiratory tract prosthesis of claim 15, wherein said ECM material comprises at least one supplemental biologically active agent.

24. The respiratory tract prosthesis of claim 23, wherein said supplemental biologically active agent comprises a I-IMG-CoA reductase inhibitor selected from the group consisting of atorvastatin, cerivastatin, fluvastatin, lovastatin, mevastatin, pitavastatin, pravastatin, rosuvastatin, and simvastatin.

25. The respiratory tract prosthesis of claim 23, wherein said supplemental biologically active agent comprises a cell selected from the group consisting of human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

26. The respiratory tract prosthesis of claim 23, wherein said supplemental biologically active agent comprises a growth factor selected from the group comprising a platelet derived growth factor (PDGF), epidermal growth factor (EGF), transforming growth factor-α (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor α (TNA-α), and placental growth factor (PLGF).

27. The respiratory tract prosthesis of claim 15, wherein said ECM material comprises at least one pharmacological agent.

28. The respiratory tract prosthesis of claim 27, wherein said pharmacological agent comprises an agent selected from the group consisting of antibiotics, anti-virals, anesthetics, analgesics and anti-inflammatories.

29. A regenerative respiratory tract prosthesis, comprising:
a tubular member comprising an extracellular matrix (ECM) composition, said ECM composition comprising acellular ECM from a mammalian tissue source,
said tubular member having an interior and exterior surface, and proximal and distal ends, said interior surface defining a conduit through said tubular member,
said tubular member being capable of transitioning from a first pre-deployment configuration, wherein said tubular member is capable of being positioned at a target position in a respiratory tract structure, to an expanded first post-deployment configuration, wherein said tubular member is capable of being disposed proximate host tissue of said a respiratory tract structure;
a first expandable anchor disposed in said conduit of said tubular member on said interior surface and proximate said proximal end; and
a second expandable anchor disposed in said conduit of said tubular member on said interior surface proximate said distal end,
said first expandable anchor and said second expandable anchor being capable of transitioning from a second pre-deployment configuration when said tubular member is in said first pre-deployment configuration, to a second post-deployment configuration, thereby causing said tubular member to assume said expanded first post-deployment configuration, and wherein said first expandable anchor and said second expandable anchor engage and support said tubular member at a first region proximate said interior surface, and wherein said first expandable anchor and said second expandable anchor are capable of positioning said exterior surface at said host tissue of said respiratory tract structure and maintain said positioning during a trigger phase, flow delivery phase, cycle phase and expiratory phase of a ventilator cycle for a temporary anchor support period of time,
said second post-deployment configuration of said first expandable anchor and said second expandable anchor comprising a circular planar shape,
said first expandable anchor and said second expandable anchor being configured to exert a diminishing supporting force on said tubular member in second post-deployment configuration that diminishes to 0 lb. after said temporary anchor support period of time.

30. The respiratory tract prosthesis of claim 29, wherein said temporary anchor support period of time is during a process of tissue regeneration.

33. The respiratory tract prosthesis of claim 29, wherein said first expandable anchor and said second expandable anchor comprise a biocompatible shape memory alloy.

34. The respiratory tract prosthesis of claim 33, wherein said biocompatible shape memory alloy comprises a nickel-titanium alloy.

35. The respiratory tract prosthesis of claim 29, wherein said first expandable anchor and said second expandable anchor each anchors comprise a respective single anchor ring.

37. The respiratory tract prosthesis of claim 29, wherein said mammalian tissue source is selected from the group consisting of small intestine submucosa (SIS), urinary bladder submucosa (UBS), stomach submucosa (SS), subcutaneous extracellular matrix, gastrointestinal extracellular matrix, placental extracellular matrix, omentum extracellular matrix, cardiac extracellular matrix, kidney extracellular matrix, pancreas extracellular matrix, lung extracellular matrix, mesothelial tissue and combinations thereof.

38. The respiratory tract prosthesis of claim 37, wherein said ECM composition comprises at least one supplemental biologically active agent.

39. The respiratory tract prosthesis of claim 38, wherein said supplemental biologically active agent comprises a HMG-CoA reductase inhibitor selected from the group consisting of atorvastatin, cerivastatin, fluvastatin, lovastatin, mevastatin, pitavastatin, pravastatin, rosuvastatin, and simvastatin.

40. The respiratory tract prosthesis of claim 38, wherein said supplemental biologically active agent comprises a cell selected from the group consisting of human embryonic stem cells, fetal cardiomyocytes, myofibroblasts, mesenchymal stem cells, autotransplated expanded cardiomyocytes, adipocytes, blood stem cells, myoblasts, adult stem cells, bone marrow cells, parenchymal cells, epithelial cells, endothelial cells, mesothelial cells, fibroblasts, osteoblasts, chondrocytes, bone-marrow derived progenitor cells, myocardial cells.

41. The respiratory tract prosthesis of claim 38, wherein said supplemental biologically active agent comprises a growth factor selected from the group comprising transforming growth factor-α (TGF-α), transforming growth factor beta (TGF-β), fibroblast growth factor-2 (FGF-2), vascular epithelial growth factor (VEGF), hepatocyte growth factor (HGF), insulin-like growth factor (IGF), and placental growth factor (PLGF).

42. The respiratory tract prosthesis of claim 29, wherein said ECM composition comprises at least one pharmacological agent selected from the group consisting of an antibiotic, anti-viral, anesthetic, analgesic and anti-inflammatory.
